SEQUENCE LISTING (1 sequence)

SEQ ID NO: 1 - DNA, 45 bp, Artificial
  HindIII site and the T7 gene 10 rbs
cccaagctttttaagaaggagatatacatatgagtaaaggagaag

(19) United States
(12) Patent Application Publication
(10) Pub. No.: US 2009/0123512 A1
(43) Pub. Date: May 14, 2009

(54) Quorum Sensing Modulators

(76) Inventors: Ute Muh, Iowa City, IA (US); Brian Hare, Cambridge, MA (US); Eric Olson, Belmont, MA (US); E. Peter Greenberg, Seattle, WA (US)

Correspondence Address: Miller Canfield Paddock And Stone PLC, 444 W. Michigan Avenue, Kalamazoo, MI, 49007, US

(21) Appl. No.: 11/795,620

(22) PCT Filed: Jan. 20, 2006
(86) PCT No.: PCT/US06/02256

§ 371 Date: May 13, 2008

Related U.S. Application Data
(60) Provisional application No. 60/646,061, filed on Jan. 21, 2005.

(51) Int. Cl. A61F 2/04; A01N 37/18; A61K 31/165; A61K 9/00; A61F 6/14; A61P 31/04

(52) U.S. Cl. 424/423; 514/621; 424/400; 424/430; 424/429

(57) ABSTRACT

Compounds described herein are useful in modulating bacterial quorum sensing.

### CLAIM OF PRIORITY

This application claims the benefit of U.S. provisional application 60/646,061, filed on Jan. 21, 2005, which is hereby incorporated by reference.

### TECHNICAL FIELD OF THE INVENTION

The present invention relates to compounds useful as modulators of quorum sensing.

### BACKGROUND OF THE INVENTION

Quorum sensing refers to the regulation of virulence genes in response to cell density. When the cell density or population of bacteria has achieved a particular level, specific genes are activated or repressed. Many of these affect virulence factors or mediate survival of the bacteria in the host.

Quorum sensing is mediated by a signal molecule that binds to a cognate transcriptional activator to cause either up-regulation or repression of genes that increase virulence factors, which include exotoxins, proteases, alginates, lipopolysaccharides, pyocyanin and rhamnolipids. At low bacterial cell density, the concentration of the signaling molecule does not activate the virulence genes, while at higher bacterial density, the concentration of the signaling molecule reaches a critical threshold to activate virulence genes.

In Gram negative bacteria, the signal molecule is an acylated homoserine lactone (AHSL), often referred to as the autoinducer, which interacts with a protein of a quorum regulon. A quorum regulon includes two proteins, the autoinducer synthase (the I protein) and the regulator (the R protein), which, upon binding of the autoinducer, activates the transcription of numerous genes. In Pseudomonas aeruginosa, two quorum regulons have been identified. One quorum regulon is known as the LasIR system and is mediated by a 3-oxo-dodecanoyl homoserine lactone (3-oxo-C12-HSL) signal molecule. The other quorum regulon is known as the Rh1IR system and is mediated by a butyryl homoserine lactone (C4-HSL) signal molecule.

Quorum sensing plays a role in the formation of bacterial biofilms, a form of growth in which bacteria are attached to a surface and encased in a matrix. These bacterial biofilms frequently show reduced sensitivity to treatment with antibiotics and biocides. Studies with animal models have shown that strains with inactivated quorum sensing genes show reduced virulence. Disrupting quorum sensing may interfere with the ability of bacteria to form robust biofilms and thus render the bacteria more sensitive to antibacterial agents and the host's immune response. Thus, there is a need to identify and develop compounds that are useful as modulators of quorum sensing.

### SUMMARY

The present invention addresses this need by identifying compounds that are useful as modulators of quorum sensing to affect the virulence of bacteria and thus their sensitivity to antibacterial agents or a host's immune system.

In one aspect, the invention features a method of modulating bacterial quorum sensing by contacting bacteria with a compound of formula I

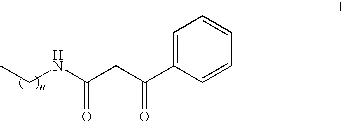

wherein

n is 3 to 13.

In another aspect, the invention features a pharmaceutical composition including a pharmaceutical carrier and a compound of formula I.

In still another aspect, the invention features a method of treating or reducing the severity of a bacterial infection in a subject by administering, to the subject, a therapeutically effective amount of a compound of formula I.

In still another aspect, the invention provides an implantable or indwelling device including a compound of formula I.

### DETAILED DESCRIPTION OF THE DRAWINGS

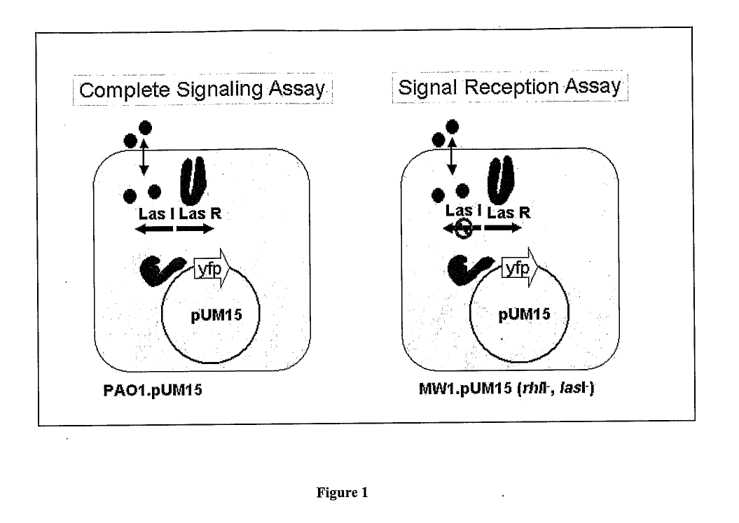

FIG. 1 illustrates the signaling assays in P. aeruginosa.

### DETAILED DESCRIPTION OF THE INVENTION

### I. Definitions

As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito, 1999, and “March's Advanced Organic Chemistry”, 5^{th} Ed., Ed.: Smith, M. B. and March, J., John Wiley & Sons, New York, 2001, the entire contents of which are hereby incorporated by reference.

The term “modulating” as used herein means increasing or decreasing, e.g. activity, by a measurable amount. Compounds that modulate quorum sensing by increasing the activity of the virulence genes are called agonists. Compounds that modulate quorum sensing by decreasing the activity of the virulence genes are called antagonists. Without being bound by any theory, it is believed that an agonist interacts with a quorum sensing receptor to increase the ability of the receptor to modulate relevant gene expression while an antagonist interacts with a quorum sensing receptor to decrease the ability of the receptor to modulate relevant gene expression.

The phrase “treating or reducing the severity of a quorum sensing mediated condition” refers both to treatments for conditions that are directly caused by quorum sensing, such as a primary bacterial infection, and alleviation of symptoms caused by bacterial infections in conditions that are not directly caused by quorum sensing. Examples of conditions caused by primary bacterial infections include septicemia and corneal infections. Examples of conditions whose symptoms may be affected by quorum sensing include, but are not limited to, cystic fibrosis, AIDS and burns. Other conditions related to quorum sensing include vibriosis, e.g., hemorrhagic septicaemia in fish, and plant rot.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### II. Description of Compounds:

Compounds useful for modulating quorum sensing have the structure shown in formula I

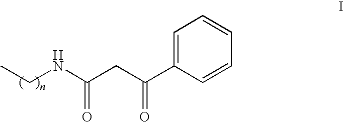

wherein

n is 3 to 13.

In one embodiment, n is 5 to 10. In a further embodiment, n is 8.

### III. General Synthetic Methodology

The compounds useful for modulating quorum sensing may be prepared in general by methods known to those skilled in the art for analogous compounds. The compounds of formula I and starting materials useful for producing the compounds of formula I are commercially available from chemical reagent supply companies, such as, Aldrich Chemicals Co., Sigma Chemical Company, and the like. Compounds also can be prepared by those of ordinary skill in art following procedures set forth in references such as, “Fieser and Fieser's Reagents for Organic Synthesis”, Volumes 1-15, John Wiley and Sons, 1991; “Rodd's Chemistry of Carbon Compounds”, Volumes 1-5 and Supplements, Elsevier Science Publishers, 1989; and “Organic Reactions”, Volumes 1-40, John Wiley and Sons, 1991.

### IV. Uses, Formulations, Compositions and Administration

The present invention includes within its scope pharmaceutically acceptable prodrugs of the compounds of the present invention. A “pharmaceutically acceptable prodrug” means any pharmaceutically acceptable salt, ester, salt of an ester, or other derivative of a compound of the present invention which, upon administration to a recipient, is capable of providing (directly or indirectly) a compound of this invention or an active metabolite or residue thereof. Preferred prodrugs are those that increase the bioavailability of the compounds of this invention when such compounds are administered to a mammal or which enhance delivery of the compound to a biological compartment relative to the non-prodrug form of the compound.

The term “pharmaceutically acceptable carrier, adjuvant, or vehicle” refers to a carrier, adjuvant, or vehicle that does not destroy the pharmacological activity of the compound with which it is formulated and which is not toxic to the subject to which the compound is to be administered. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., calcium or magnesium), ammonium and N⁺(C₁₋₄ alkyl)₄ salts or salts of lysine and arginine. This invention also envisions the quaternization of any basic nitrogen-containing groups of the compounds disclosed herein. Water or oil-soluble or dispersible products may be obtained by such quaternization. Other salts can be found in “Practical Process, Research, & Development,” Anderson, Neal G., Academic Press, 2000, the contents of which are incorporated herein by reference.

The compositions of the present invention may be administered orally, parenterally, by inhalation spray, topically, rectally, intermuscularly, subcutaneously, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In certain embodiments, the pharmaceutically acceptable compositions of this invention are formulated for oral administration.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds of formula I may also be delivered by implantation (e.g., surgically), such as with an implantable or indwelling device. An implantable or indwelling device may be designed to reside either permanently or temporarily in a subject. Examples of implantable and indwelling devices include, but are not limited to, contact lenses, central venous catheters and needleless connectors, endotracheal tubes, intrauterine devices, mechanical heart valves, pacemakers, peritoneal dialysis catheters, prosthetic joints, such as hip and knee replacements, tympanostomy tubes, urinary catheters, voice prostheses, stents, delivery pumps, vascular filters and implantable control release compositions. Biofilms can detrimental to the health of patients with an implantable or indwelling medical device because they introduce an artificial substratum into the body and can cause persistent infections. Thus, providing a compound of formula I in or on the implantable or indwelling device can prevent or reduce the production of a biofilm. In addition, implantable or indwelling devices may be used as a depot or reservoir of a compound of formula I. Any implantable or indwelling device can be used to deliver the compound provided that 1) the device, compound and any pharmaceutical composition including the compound are biocompatible, and 2) that the device can deliver or release an effective amount of the compound to confer a therapeutic effect on the treated patient.

Delivery of therapeutic agents via implantable or indwelling devices is known in the art. See for example, “Recent Developments in Coated Stents” by Hofma et al. published in Current Interventional Cardiology Reports 2001, 3:28-36, the entire contents of which, including references cited therein, are incorporated herein. Other descriptions of implantable devices can be found in U.S. Pat. Nos. 6,569,195, 6,835,387 and 6,322,847 and U.S. Patent Application Numbers 2004/0044405, 2004/0018228, 2003/0229390, 2003/0225450, 2003/0216699 and 2003/0204168, each of which is incorporated herein in its entirety.

In some embodiments, the implantable device is a stent. In one specific embodiment, a stent can include interlocked meshed cables. Each cable can include metal wires for structural support and polyermic wires for delivering the therapeutic agent. The polymeric wire can be dosed by immersing the polymer in a solution of the therapeutic agent. Alternatively, the therapeutic agent can be embedded in the polymeric wire during the formation of the wire from polymeric precursor solutions.

In other embodiments, implantable or indwelling devices can be coated with polymeric coatings that include the therapeutic agent. The polymeric coating can be designed to control the release rate of the therapeutic agent. Controlled release of therapeutic agents can utilize various technologies. Devices are known that have a monolithic layer or coating incorporating a heterogeneous solution and/or dispersion of an active agent in a polymeric substance, where the diffusion of the agent is rate limiting, as the agent diffuses through the polymer to the polymer-fluid interface and is released into the surrounding fluid. In some devices, a soluble substance is also dissolved or dispersed in the polymeric material, such that additional pores or channels are left after the material dissolves. A matrix device is generally diffusion limited as well, but with the channels or other internal geometry of the device also playing a role in releasing the agent to the fluid. The channels can be pre-existing channels or channels left behind by released agent or other soluble substances.

Erodable or degradable devices typically have the active agent physically immobilized in the polymer. The active agent can be dissolved and/or dispersed throughout the polymeric material. The polymeric material is often hydrolytically degraded over time through hydrolysis of labile bonds, allowing the polymer to erode into the fluid, releasing the active agent into the fluid. Hydrophilic polymers have a generally faster rate of erosion relative to hydrophobic polymers. Hydrophobic polymers are believed to have almost purely surface diffusion of active agent, having erosion from the surface inwards. Hydrophilic polymers are believed to allow water to penetrate the surface of the polymer, allowing hydrolysis of labile bonds beneath the surface, which can lead to homogeneous or bulk erosion of polymer.

The implantable or indwelling device coating can include a blend of polymers each having a different release rate of the therapeutic agent. For instance, the coating can include a polylactic acid/polyethylene oxide (PLA-PEO) copolymer and a polylactic acid/polycaprolactone (PLA-PCL) copolymer. The polylactic acid/polyethylene oxide (PLA-PEO) copolymer can exhibit a higher release rate of therapeutic agent relative to the polylactic acid/polycaprolactone (PLA-PCL) copolymer. The relative amounts and dosage rates of therapeutic agent delivered over time can be controlled by controlling the relative amounts of the faster releasing polymers relative to the slower releasing polymers. For higher initial release rates the proportion of faster releasing polymer can be increased relative to the slower releasing polymer. If most of the dosage is desired to be released over a long time period, most of the polymer can be the slower releasing polymer. The device can be coated by spraying the device with a solution or dispersion of polymer, active agent and solvent. The solvent can be evaporated, leaving a coating of polymer and active agent. The active agent can be dissolved and/or dispersed in the polymer. In some embodiments, the co-polymers can be extruded over the device.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the modulator can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention. For instance, compounds of formula I may be administered in combination with other antibacterial agents. The compounds of formula I may be administered with other antibacterial agents in any order such as sequentially or simultaneously. As used herein, additional therapeutic agents that are normally administered to treat or prevent a particular disease, or condition, are known as “appropriate for the disease, or condition, being treated.”

According to one embodiment, the compounds of formula I are agonists or antagonists of quorum sensing. Antagonist compounds of formula I can be used alone to treat bacterial infections or in combination with other antibacterial agents. Agonist compounds of formula I are useful in studying quorum sensing, e.g., in developing model systems for bacterial infections that can be used for testing antagonist quorum sensing compounds and/or other antibacterial compounds. Agonist compounds of formula I are also useful for habitat remediation. Compounds of formula I can be used to promote the formation of biofilms for water treatment plants, waste water treatment plants and private septic systems that remove pathogens and reduce the amount of organic matter in the water or waste water through interaction with biofilms

Agonist compounds of formula I could also be used to drive heterologous gene expression in bacterial and eukaryotic systems. A Heterologous gene expression system may include a lasR or homolog and a quorum sensing promoter that regulates the gene of interest. The agonist could be used with the heterologous gene expression system to drive the over expression or time induction of the target gene. Additionally, agonist compounds of formula I could be used as a therapeutic, protecting a subject against a bacterial infection, by prematurely inducing a quorum sensing controlled response and thereby rendering a bacterial population less virulent and/or more susceptible to an antibiotic regimen or the host immune response. The compound may be administered prophylactically or at the onset of an infection.

In some embodiments, the compounds of formula I are agonists or antagonists of quorum sensing in Gram negative bacteria. In other embodiments, the compounds are agonists or antagonists of quorum sensing in Burkholderia cepacia, Burkholderia mallei, Burkholderia pseudomallei, Serratia liquifaciens, Yersinia enterocholitica, Yersinia pseudotuberculosis, Aeromonas hydrophila, Aeromonas salmonicida, Erwinia carotovora, Erwinia chrysanthemi, Pantoea stewartii and Pseudomonas aeruginosa. In other embodiments, the compounds of formula I are agonists or antagonists of quorum sensing in Pseudomonas aeruginosa.

In order that the invention described herein may be more fully understood, the following examples are set forth. It should be understood that these examples are for illustrative purposes only and are not to be construed as limiting this invention in any manner.

All references cited above are incorporated herein by reference.

Certain embodiments of the compounds of formula I are shown below. The following examples are illustrative of the compounds of formula I and are not meant to be limiting.

### EXAMPLES

### Example 1

### Assays for Determining Activation or Inhibition of Quorum Sensing

Assays were developed to characterize and dissect signaling events in P. aeruginosa. The signaling events are generally illustrated in FIG. 1. Two types of assays are performed on the compounds of formula I, the Complete Signaling Assay (CSA) and the Signal Reception Assay (SRA).

The CSA yields a fluorescent signal reporting both signal synthesis and signal reception. The SRA yields a fluorescent signal due to signal reception. Compounds affecting signal reception can be detected by SRA. The SRA can detect compounds that modulate quorum sensing by inhibiting signal reception or activating signal reception. Inhibitor compounds are identified by performing the SRA with exogenous autoinducer at a concentration of half maximal activation and detecting changes in fluorescence indicative of inhibition of signal reception. Activator compounds are identified by performing the SRA in the absence of exogenous autoinducer.

The biological assays, CSA and SRA, useful for identifying modulators of quorum sensing are described below and in more detail in U.S. Serial No. 894,710, filed on Jul. 19, 2004, which is incorporated by reference in its entirety.

### I. Materials and Methods

### Bacterial Strains, Plasmids and Culture Media.

Bacterial strains and plasmids described below in Table 1 may be used to produce materials for use in quorum sensing assays. Unless otherwise noted, cultures were grown in Luria-Bertani (LB) broth or agar with lowered salt (4 g/L NaCl) and containing the appropriate antibiotics for plasmid screening and maintenance (300 μg/mL carbenicillin for P. aeruginosa and 100 μg/mL ampicillin, 20 μg/mL kanamycin, 20 μg/mL tetracycline for E. coli).

TABLE 1
Strains and plasmids

| | |
|---|---|
| Strain | Description |

Pseudomonas aeruginosa

| | |
|---|---|
| PAO1 | wild type |
| PAO lasR rhlR | (ΔlasR::Tc^{r} ΔrhlR::Gm^{r}) of PAO1 |
| PAO-MW1 | (rhlI::Tn501 lasI::tetA) of PAO1 |

Escherichia coli

| | |
|---|---|
| DH5α | F⁻ Φ80dlacZΔM15 Δ(lacZYA-argF)U169 endA1 recA1 hsdR17 deoR gyrA96 |
| | thi-1 relA1 supE44 |
| MG4 | Δ(argF-lac)U169 zah-735::Tn10 recA56 srl::Tn10 |
| VJS533 | ara Δ(1ac-proAB) rpsL φ80lacZ ΔM15 recA56 |
| Top 10F′ | F′{lacI^{q}, Tn10(Tet^{R})} mcrA Δ(mrr-hsdRMS-mcrBC) Φ80lacZΔM15 ΔlacX74 |
| | recA1 araD139 Δ(ara-leu)7697 galU galK rpsL (Str^{R}) endA1 nupG |

Plasmids

| | |
|---|---|
| pKDT17 | lasB::lacZ translational fusion and plac::lasR in pTS400; Ap^{r} |
| pECP61.5 | rhlA::lacZ translational fusion and ptac::rhlR in pSW205; Ap^{r} |
| pHV200I⁻ | 8.8-kb Vibrio fischeri ES114 lux regulon with inactivated luxI in pBR322; Ap^{r} |
| pQF50 | lacZ transcriptional fusion vector; Ap^{r} |
| pMW312 | pQF50 carrying rsaL promoter from −82 to +29 relative to the translational start |
| | of rsaL; Ap^{r} |
| pRSET(B) | Expression vector |
| pRSET(B)-10Bnh | yfp cloned into pRSET(B) |
| pUC18 | Cloning vector |
| pUM11 | yfp with T7gene10 rbs in pUC18 |
| pUM15 | rsaL::yfp transcriptional fusion, derived from pUM11 and pMW312; Ap^{r} |
| pPROLar.A122 | Expression vector; Kan^{r} |
| pPROLasR | lasR under control of the lac/ara-1 promoter |

### II. Plasmid Construction.

A yfp containing fragment was amplified by polymerase chain reaction (PCR) from BamHI digested pRSET(B)-10 Bnh with a forward primary primer complimentary to the first 16 bases of the open reading frame (ORF) and a reverse primer complimentary to the stop codon and the last 12 bases of the yfp ORF with an engineered AatII site 5′ to the stop codon. An engineered HindIII site and the T7gene10 rbs (CCCAAGCTTTTTAAGAAGGAGATATACATATGAGTAAAGGAGAAG) (SEQ ID NO:1) were also introduced. The resulting PCR product was ligated into AatII/HindIII and the pUC18 vector digested to yield pUM11. The rbs-yfp fragment was then excised with HindIII/ScaI and ligated into the same sites of pMW312, thereby replacing the lacZ reporter with yfp and a T7gene10 rbs. The final construct was called pUM15.

The plasmid pProlasR was constructed by ligation of a PCR product encoding the LasR polypeptide into KpnI/BamHI and digestion of pPROLar.A122. The lasR containing PCR fragment was amplified from PAO1 (Iglewski) genomic DNA using a forward primer complementary to the first 18 bases of the lasR ORF with an engineered KpnI site 5′ to the lasR start codon and a reverse primer complementary to the stop codon and the last 16 bases of the lasR ORF with an engineered BamHI site 5′ to the stop codon.

III. Quorum Sensing Activation in E. coli:

Activation of quorum sensing controlled promoters in the heterologous host E. coli was performed with MG4.pKDT17 for LasR dependent regulation (see Pearson et. al. Proc Natl Acad Sci USA 91:197-201 (1994)), with VJS533. pHV200I⁻ for LuxR dependent regulation (see Pearson et. al. Proc Natl Acad Sci USA 91:197-201 (1994)) and with DH5α.pECP61.5 for Rh1R dependent regulation (see Pearson et. al. J Bacteriol 179:5756-67 (1997)). Assays were performed as published, using synthetic acylated homoserine lactones as positive controls. To test activation of the LasR dependent rsaL promoter in E. coli, Top10F′.pUM15.pPROLasR were grown in LB buffered with 50 mM KPᵢ, pH 7.0 containing 100 μg/mL ampicillin, 20 μg/mL kanamycin, and 20 μg/mL tetracycline. After overnight growth, subcultures were inoculated at a cell density of 0.05 at 620 nm (A₆₂₀) and shaken for 1 h at 37° C. before induction of LasR expression with 100 μM isopropyl-beta-D-thiogalactopyranoside (IPTG), followed by addition of test compounds. Synthetic 3-oxo-C12-HSL was added as a positive control. Aliquots of 100 μl were dispensed into microtiter wells and incubated at 37° C., shaking and humidified for 7 h. Fluorescence was read with a SpectroFluorPlus plate reader (Tecan US, Durham, N.C.) with an excitation wavelength of 485 nm and emission wavelength of 535 nm.

### IV. Signal Reception Assay (SRA)

The plasmid pUM15 carries a YFP reporter under control of a LasR dependent quorum sensing controlled promoter, prsaL. When the plasmid is harbored by MW1, a strain lacking the ability to produce acylated homoserine lactones, the reporter is induced only in the presence of exogenous autoinducer. This is referred to as the Signal Reception Assay, as only those compounds that modulate signal reception will be detected.

Cells were grown in LB with 50 mM 3-(N-morpholino)propanesulfonic acid (MOPS) pH 7.0, 300 μg/mL carbenicillin. A single colony of MW1.pUM15 from a freshly struck plate was used to inoculate a starter culture and grown shaking at 30° C. not above an absorbance at 600 nm (A₆₀₀) of 1.5. Cells were subcultured to A₆₀₀ of 0.05 and grown shaking at 37° C. for 1-2 h. These mid-logarhithmic cells were then added to dried down 3-oxo-C12-HSL to result in a final concentration of 0.3 μM. Induced cells were pipetted into wells containing the test compound. Uninduced cells were included as a control for the assay window. When the assay was used to characterize a potential activator, no homoserine lactone was added.

### V. Complete Signaling Assay (CSA).

The plasmid pUM15 carries a YFP reporter under control of a LasR dependent quorum sensing controlled promoter, prsaL. When harbored by the wild type strain, PAO1, the reporter will be expressed as the bacteria produce the autoinducer, 3-oxo-C12-HSL, during growth. This is referred to as the Complete Signaling Assay, as events that disrupt either signal synthesis or signal reception would lower the fluorescence output.

Cells were grown in LB with 300 μg/mL carbenicillin. A single colony of PAO1.pUM15 from a freshly grown plate was used to inoculate a starter culture and grown shaking at 37° C. overnight. Cells were washed twice with medium and resuspended to a density of A₆₀₀ between 0.05 and 0.1. Resuspended cells were pipetted into wells containing test compound. A culture of MW1.pUM15 was treated in parallel and used as a control for the assay window.

### VI. Microtiter Plate Format:

Nanoplate format: Assay reagents were dispensed with the flying reagent dispenser, FRD (Vertex Pharmaceuticals Incorporated, San Diego, Calif.). An aliquot of 1.5 μL reporter culture was added to wells in 3,456-well plates (3456 plates) and grown for 8-12 h before reading fluorescence with the topography-compensating plate reader, tcPR (Vertex Pharmaceuticals Incorporated, San Diego, Calif.). DR96 format: an aliquot of 50 μL culture was added to 96 well plate and grown for 6-8 h. Fluorescence was read with a SpectroFluorPlus plate reader (Tecan US, Durham, N.C.).

In all formats, plates were sealed in a humidified container and incubated statically at 37° C. Fluorescence measurements were made with an excitation wavelength of 485 nm and emission wavelength of 535 nm. To evaluate growth in the presence of test compound, the absorbance at 620 nm was also recorded.

For large scale screening using the nanoplate format, compounds (25 nl of a 2 mM stock in 75% dimethyl sulfoxide) were preprinted into 3456 plates (one compound per well) by using the piezo sample distribution robot (Vertex Pharmaceuticals Incorporated, San Diego, Calif.). Each compound was represented once in the screen. The final concentration was 33 μM.

### VII. Expression Profiling:

All cultures were grown in LB with 50 mM MOPS pH 7.0. Cells grown to mid-logarithmic phase were used to inoculate 3 mL pre-warmed medium to an initial density of 0.01 at 600 nm, and grown in 18×150 mm borosilicate tubes containing the appropriate additions. Cultures were shaken at 250 rpm, 37° C. and grown to a final density of 2.0 at 600 nm (Cary 50, Varian). A culture volume corresponding to 2×10⁹ colony forming units was mixed with RNA Protect Bacteria reagent (Qiagen) and stored at −80° C. until sample workup. RNA was isolated and processed as described by Schuster et al. in J Bacteriol 185:2066-79 (2003). The Affymetrix Microarray Software suite (MAS) version 5.0 was used to determine transcript levels and for comparison analysis of different samples.

### Example 2

### Quorum Sensing Modulators

A compound of formula I, in which n is 8, is commercially available from Chembridge Corp. (San Diego, Calif.). The compound (compound 1) exhibited quorum sensing inhibition in the SRA described in Example 1.

### Example 3

### Effect of Compounds of Formula I on Virulence Factor Expression in Pseudomonas aeruginosa

RNA profiling of cultures grown in the presence of compound 1 showed inhibition of genes expressing virulence factors, particularly lasB, the gene for elastase and phnAB, the product of which is thought to be involved in the regulation of pyocyanin synthesis. Elastase is a metalloprotease, while pyocyanin is a redoxactive molecule that leads to the production of superoxide radicals and hydrogen peroxide, both of which contribute to the extensive tissue damage during a pseudomonal infection. Elastase activity and concentration of pyocyanin were measured directly in cultures grown in the presence and absence of the inhibitor to determine whether their levels were consistent with the RNA levels.

### I. Methods

Elastase activity was measured according to the procedure described by Pearson et al. in J. Bacteriol. 179:5756-67 (1997) and pyocyanin concentration was measured according to the procedure described by Essar et al. in J. Bacteriol. 172:884-900 (1990). Protocols were modified slightly to be consistent with the conditions of transcriptional profiling. Cultures were grown in LB amended with 50 mM MOPS pH 7.0. Mid-log cultures were either subcultured directly (MW1) or washed twice with medium (PAO1) and then subcultured to an absorbance at 600 nm (A₆₀₀) of 0.01. Cultures (1 mL) were grown in the presence of test compounds in 16 mm tubes at 37° C., 250 rpm.

For elastase activity, cultures were grown for 6 hours. Cells were removed by centrifugation and the culture supernatant filtered through a 0.2 μm filter. A 100 μL volume of filtered supernatant was added to 900 μL of 5 mg/mL Elastin Congo Red (Elastin Products Company; Owensville, Mo.) in 1 mM CaCl₂, 100 mM Tris HCL, pH 7.2. Reactions were incubated at 37° C., 250 rpm for 18 h. Reactions were stopped by the addition of 100 μL 0.12 M EDTA, undigested substrate removed through centrifugation and the absorbance at 495 nm measured.

To determine levels of pyocyanin, cultures were grown for 9.5 hours in the presence of test compounds. Pyocyanin was extracted into 1 mL chloroform, followed by a second extraction into 150 μL 0.2 N HCl. The absorbance of this solution was measured at 520 nm.

### II. Results: Pyocyanin Production.

Wild type P. aeruginosa PAO1 was grown in the presence of 100 μM of Compound 1. The compound significantly reduced the concentration of pyocyanin produced. This was most likely due to inhibition of the phnAB genes.

### III. Results Elastase Production

MW1 had no measurable elastase activity in the supernatant when grown in the absence of exogenous signal. Addition of half-maximal 3-oxo-C12-HSL (0.3 μM) restored about one third of the achievable activity. If 25 μM C4-HSL was also added, maximal elastase activity was measured. The presence of saturating amounts of 3-oxo-C12-HSL (25 μM) in addition to saturating C4-HSL (25 μM) did not increase elastase activity further. Under those conditions when 3-oxo-C12-HSL was non-saturating, inhibition by 100 μM Compound 1 on the production of elastase could be observed. When wild type PAO1 was grown in the presence of 100 μM Compound 1, significant reduction in elastase production was also observed. Inhibition of elastase production by Compound 1 correlates with inhibition of lasB transcription seen in microarray studies.

What is claimed is:

1. A method of modulating bacterial quorum sensing, comprising contacting bacteria with a compound of formula I 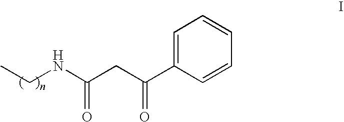
wherein
n is 3 to 13.

2. The method of claim 1, wherein n is 8.

3. A pharmaceutical composition comprising a pharmaceutical carrier and a compound of formula I 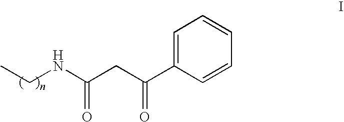
wherein
n is 3 to 13.

4. The pharmaceutical composition of claim 3, wherein n is 8.

5. A method of treating or reducing the severity of a bacterial infection in a subject, comprising administering to the subject a therapeutically effective amount of a compound of formula I 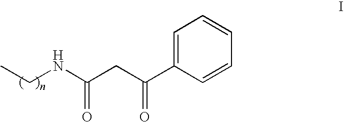
wherein
n is 3 to 13.

6. The method of claim 5, wherein n is 8.

7. An implantable or indwelling device comprising a compound of formula I 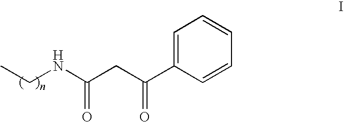
wherein
n is 3 to 13.

8. The implantable or indwelling device of claim 7 further comprising a device coating, wherein the device coating includes the compound of formula I.

9. The implantable or indwelling device of claim 7, wherein the device is a contact lens, a catheter, a needleless connector, an endotracheal tube, an intrauterine device, a mechanical heart valve, a pacemaker, a prosthetic joint, a tympanostomy tube, a voice prosthesis, a stent, a delivery pump, or a vascular filter

10. The implantable or indwelling device of claim 7, wherein n is 8.

11. The implantable or indwelling device of claim 7, wherein the compound activates quorum sensing.

12. The implantable or indwelling device of claim 7, wherein the compound inhibits quorum sensing.
